(19) United States
(12) Patent Application Publication
(10) Pub. No.: US 2007/0010777 A1
(43) Pub. Date: Jan. 11, 2007

(54) MEDICAL TAPE AND BANDAGES

(75) Inventors: Wayne K. Dunshee, Maplewood, MN (US); Craig E. Hamer, Woodbury, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 11/428,040

(22) Filed: Jun. 30, 2006

Related U.S. Application Data
(60) Provisional application No. 60/696,863, filed on Jul. 6, 2005.

(51) Int. Cl. A61F 15/00; A61F 13/00

(52) U.S. Cl. 602/54; 602/59

(57) ABSTRACT

Medical article such as medical tapes and bandages comprising a backing, a pressure sensitive adhesive on at least a portion of one side of the backing, and in the case of bandages, an absorbent pad.

### CROSS REFERENCE TO RELATED APPLICATION

This application claims the benefit of U.S. Provisional Patent Application No. 60/696,863, filed Jul. 6, 2005.

### FIELD

This invention relates to pressure-sensitive medical tapes and bandages suitable for use on humans and animals.

### BACKGROUND

Pressure-sensitive medical tapes and bandages suitable for use on humans and animals have been made in a variety of configurations, some optimized for difficult to adhere locations or conditions, e.g., wet environments, across knuckles of the hand, bending and stretching, and so forth.

Such tapes and bandages have been made with a variety of adhesives.

Medical adhesive bandages typically include a backing, an absorbent pad, and a pressure sensitive adhesive to maintain the medical adhesive bandage in place. The bandages are typically used to cover cuts, scrapes and other skin conditions. U.S. Pat. No. 5,939,339 (Delmore et al.) is one example of such bandages. U.S. Pat. No. 4,366,814 (Riedel) is an example of a backing material that can be used on medical tapes and bandages.

A need exists for medical adhesive tapes and bandages that can be easily applied by lay persons and that will adhere well to skin and hair, preferably also under sweaty and wet conditions.

As referred to herein, “bandage” or “bandages” will be understood to refer to medical adhesive bandages.

### SUMMARY

The present invention provdes medical article such as medical tapes and bandages comprising a backing having first and second major surfaces, a pressure sensitive adhesive on at least a portion of the first major surface of the backing (i.e., the side to be disposed to the wearer), and in the case of bandages, an absorbent pad.

The medical articles of the present invention, which are typically in tape or bandage form, can be made with a visually similar appearance to duct tape.

### DETAILED DESCRIPTION OF ILLUSTRATIVE EMBODIMENTS

As described above, the present invention provides pressure-sensitive adhesive medical articles such as tapes and bandages.

In general, medical articles of the invention will comprise a backing with two major surfaces, i.e., an outer major surface and an inner major surface, a reinforcing scrim, and a layer of pressure-sensitive adhesive disposed on the inner major surface thereof.

In the case of a tape, the article will typically consist essentially of a backing and an adhesive layer as described herein.

In the case of a bandage, the article will typically further comprise an absorbent pad disposed on the inner major surface.

Backing

Typically backings of medical articles of the invention will be selected for an appearance and properties in common with duct tape. Illustrative examples of backing materials include polyethylene, other polyolefins, and polyurethane polymers and blends of such materials.

Examples of suitable polymers for the backing include thermoplastic polymers, preferably thermoplastic polymers that can be extruded (e.g., using a blown film or cast film extrusion process). Representative examples of thermoplastic polymers include polyolefins (e.g., low density polyethylene (LDPE), high density polyethylene (HDPE), linear low density polyethylene (LLDPE), polypropylene and polybutylene), polyester, copolyester, polyamide (e.g., nylon), polyvinyl chloride, polycarbonate, polytetrafluoroethylene, and mixtures thereof. One example of a suitable alternative is low density polyethylene commercially available under the trade designation “LD 129” from ExxonMobil Chemicals.

For example, an approximately 0.0025 inch aluminum pigmented polyethylene film can be used if desired. Typically the tape is reinforced with a polyester scrim to enhance its strength properties. Selection of scrim material may be made to achieve desired properties, e.g., to provide elasticity in the longitudinal direction coupled with desired tear strength properties.

It will typically be preferred to perforate at least a portion of the backing with holes or channels that extend completely through the backing to facilitate passage of moisture from the wearer through the medical article. The holes may be arranged in any desired manner.

Scrim

Articles of the invention may comprise a reinforcing scrim. The scrim may be embedded withint the backing or be bonded to the first major surface thereof.

The reinforcing scrim may, if desired, be a non-woven scrim. More typically, however, the scrim will typically be a woven or knitted scrim that is readily tearable in at least the cross-web direction. The scrim includes a plurality of warp elements oriented in the machine or down-web direction of the scrim and a plurality of weft elements that are oriented in the cross-web direction of the scrim. The warp elements are typically arranged such that they are uniformly spaced and substantially parallel to one another. Similarly, the weft elements are typically arranged such that they are uniformly spaced and substantially parallel to the other weft elements. Relative to one another, the warp elements are substantially perpendicular to the weft elements. The warp and weft elements may comprise filaments, multi-filaments (i.e., filament bundles), yarns, threads, or a combination thereof. The elements may be twisted or textured, for example, to add loft and/or stretch to the scrim.

The warp and weft elements are typically combined by a weaving or knitting process to form the scrim. Examples include plain weaves (i.e., one-over-one weaves), drill weaves (i.e., two-over-one weave), twill weaves (i.e., three-over-one weave), and sateen weaves (i.e., four-over-one weave of the warp yarns over the weft yarns).

The scrims typically have a warp density ranging from about 9 to about 60 warp elements per inch (3.5 to 23 warp elements per cm), more typically ranging from about 12 to about 30 warp elements per inch (5 to 12 warp elements per cm). The weft density typically ranges from about 5 to about 30 weft elements per inch (2 to 12 weft elements per cm), more typically ranging from about 7 to about 15 weft elements per inch (3 to 6 weft elements per cm). Scrims having a loose weave (i.e., low warp/weft density) provide a low cost construction generally having a lower tensile strength. Scrims having a tighter weave (i.e., higher warp/weft density) result in a higher cost construction generally having a higher tensile strength. Unbalanced weave constructions are useful, for example, when a particular performance property is desired in one direction. For example, it is common to have a higher scrim density in the warp direction than in the weft direction in order to provide increased tensile strength in the longitudinal direction of the tape.

The denier of the warp and weft elements are typically selected to provide a tape that can be easily hand torn in the transverse direction, the longitudinal direction, or both. If the denier is too large, the tape will not tear easily. If the denier is too small, the warp and weft elements may not effectively control the direction of tear. That is, the warp and weft elements may be too weak to direct the tear in a substantially straight line. Suitable denier will depend upon the type of element (i.e., filament, multi-filament, yarn, etc.) and the strength of the material used. Typically, the warp elements have a denier ranging from about 40 to about 150, more preferably ranging from about 70 to about 110. Typically, the weft elements have a denier ranging from about 70 to about 300, more preferably ranging from about 110 to about 200.

The scrim may be made of natural materials or synthetic materials. Illustrative examples of natural materials include cotton, silk, hemp, flax, and combinations thereof. Examples of synthetic materials include polyester, acrylic, polyolefin (e.g., polyethylene and polypropylene), nylon and combinations thereof. Natural materials and synthetic materials may also be combined, for example, in a 65/35 polyester/cotton blend or a 80/20 polyester/cotton blend.

Representative examples of commercially available scrims include those manufactured by American Fiber & Finishing, Inc. (Newberry, S.C.) having a 70 denier multi-filament polyester warp yarn and a 150 denier multi-filament polyester weft yarn woven in a one-over-one weave and having a scrim density of 30 warp/inch×10 weft/inch (12 warp/cm×4 weft/cm), 30 warp/inch×8 weft/inch (12 warp/cm×3 weft/cm), 25 warp/inch×7 weft/inch (10 warp/cm×3 weft/cm), 22 warp/inch×7 weft/inch (9 warp/cm×3 weft/cm), or 20 warp/inch×6 weft/inch (8 warp/cm×2 weft/cm).

Fibers used to make the reinforcing layer can be individual or in bundles, and those fibers and/or bundles of fibers can be randomly disposed to form a non-woven reinforcing layer, or can be disposed in a rectangular pattern with space between the fibers or bundles to form a reinforcing layer that can be manually broken at the intersections between the woven fibers or bundles when the backing layer is torn to guide straight line tearing of the tape in either of two directions at right angles to each other. When the fibers are in bundles they should be disposed so that they can easily be wetted by the adhesive (i.e., fibers in bundles that are twisted, knitted, tacked at close intervals or are in non-woven layers have been found to be more difficult to wet than when the fibers are loosely laid in the bundles) and should have diameters that are sufficiently small so that almost all of the fibers in the bundles will be wetted by the adhesive when the tape is applied to a substrate. The deniers of the individual fibers in bundles of fibers forming the scrim should generally be in the range of about 0.10 to about 15 denier, should preferably be in the range of about 0.75 to about 10 denier, and should most preferably be in the range of about 1 to about 5 denier; and the total of the deniers of the individual fibers in the bundles should generally be less than about 500, should preferably be less than about 300, and should most preferably be less than about 250.

Adhesive

The preferred pressure sensitive adhesives which can be used in the medical adhesive articles of the present invention are adhesives which are known to be useful for application to skin. The thickness of the adhesive layer is greater than is typically employed in medical tapes and bandages. Typically the adhesive layer will be between at least about 20 grains per 24 square inches area and about 40 grains per 24 square inches area. Adhesive layers which are thinner than this range may tend to be more likely to leave residue when resultant medical articles are removed from a wearer whereas using thicker adhesive layers may tend to merely entail greater cost with no beneficial change in performance. One important aspect of the present invention is the discovery that using relatively thicker layers of known adhesive formulations can yield medical articles that exhibit lower than expected levels of residue when they are peeled from the skin.

A preferred class of adhesives are those disclosed in U.S. Pat. No. 6,441,092. One example is a blend of 85 weight percent of 2-ethylhexyl acrylate/acrylic acid/ABP (96.5/3.5/0.05 weight ratio) and 15 weight percent Avalure AC 210 Acrylate copolymer. Adhesives containing from about 5 to about 20 weight percent of such hydrophilic materials provide a good balance of desired moisture permeability without unduly softening the adhesive layer to yield undesirable levels of residue. These adhesives can provide a desirable “breathability” that permits pass through of sweat from the skin surface, making tapes and bandages of the invention more comfortable when worn in hot conditions.

Other illustrative examples of useful adhesives include those described in U.S. Pat. No. 4,112,177 (particularly the tackified acrylate “skin layer adhesive” described in Example 1), U.S. Pat. No. 5,648,166, acrylate copolymers as described in U.S. Pat. No. RE 24,906, and a 70:15:15 isooctyl acrylate:ethyleneoxide acrylate:acrylic acid terpolymer, as described in U.S. Pat. No. 4,737,410 (see Example 31). Other illustrative examples of useful adhesives are described in U.S. Pat. Nos. 3,389,827, 4,112,213, 4,310,509, 4,323,557, and 6,497,949.

If desired, medicaments or antimicrobial agents may be included in the adhesive, for example, as described in U.S. Pat. Nos. 4,310,509 and 4,323,557.

The pressure sensitive adhesives 24 preferably transmit moisture vapor to increase patient comfort. While moisture vapor transmission can be achieved through the selection of an appropriate adhesive, it is also contemplated in the present invention that other methods of achieving a high relative rate of moisture vapor transmission may be used, such as pattern coating the adhesive on the backing, as described in U.S. Pat. No. 4,595,001.

In the preferred embodiments according to the present invention, the choice of adhesives is limited to those that are safe to use on human or animal skin, and preferably to those that are of the class known as “hypoallergenic” adhesives. The preferred acrylate copolymers are adhesives of this class.

The adhesive layer may be of a thickness similar to those known in the art, but it is typically preferred that the adhesive layer be thicker. For example, an adhesive layer of the above-described blend of 2-ethylhexyl acrylate/acrylic acid/ABP and Avalure™ AC 210 Acrylate copolymer coated at about 29 grains per 24 square inches of tape (a coating weight that is two to three times that typically used for bandages) has been found useful.

Absorbent Pad

Bandages of the invention will further comprise an absorbent pad.

The pad can be provided in any desired configuration, e.g., a rectangular portion that is smaller than the overall dimensions of the backing and preferably centered on the inner major surface of the backing. The pad may reach to one or both sides of the backing or may be completely surrounded by the adhesive as desired. It will be understood that, although the pad is typically generally rectangular and centered on the backing, it can take any appropriate shape and/or the pad can be located off-center on the backing as desired.

The pad is typically absorbent, and can be manufactured from a number of materials including but not limited to, woven or nonwoven cotton, rayon, nonwovens, hydrocolloids, foams, and combinations thereof.

If desired, the pad may also contain a number of substances, including antimicrobial agents, anesthetics, anti-itch agents, drugs for transdermal drug delivery, chemical indicators to monitor hormones or other substances in a patient, combinations thereof and the like.

Size/Configuration/Packaging

Medical articles of the invention may be made in any desired configuration and size. For instance, tapes are often made in generally rectangular strips ranging in selected width, e.g., 0.5 inch, 1 inch, 2 centimeter, etc. and length, e.g., precut strips on the order of 2 to 3 inches up to long lengths of 60 yards or meters or more (with the longer lengths commonly wound into roll form). Although tapes are often made with substantially uniform width and straight sides, it will be understood that a tape of the invention may be made with varying width and/or non-straight sides if desired.

Medical articles of the invention may be packaged in many desired configurations. In the case of tape, they will perhaps most commonly be packaged as precut strips or wound into roll form. In the case of bandages, they will preferably be packaged in sterile packages. If desired, the inner side of the article, i.e., the surface to be applied to the skin of a wearer, may be covered with a removable liner.

All patents, patent applications, and publications cited herein are each incorporated herein by reference in their entirety, as if individually incorporated by reference. Various modifications and alterations of this invention will become apparent to those skilled in the art without departing from the scope of this invention, and it should be understood that this invention is not to be unduly limited to the illustrative embodiments set forth herein.

What is claimed is:

1. A medical article comprising a backing having first and second major surfaces and a pressure sensitive adhesive on at least a portion of said first major surface wherein.

2. The article of claim 1 wherein said backing comprises one or more polyolefin polymers, one or more polyurethane polymers, or blends of such materials.

3. The article of claim 1 wherein said backing comprises an aluminum pigmented polyethylene film.

4. The article of claim 1 wherein said backing is perforated.

5. The article of claim 1 wherein said backing further comprises a scrim.

6. The article of claim 1 wherein said adhesive is between about 20 grains and 40 grains per 24 square inch area.
